(19) United States
(12) Patent Application Publication
(10) Pub. No.: US 2021/0153779 A1
(43) Pub. Date: May 27, 2021

(54) SENSOR UNIT AND BED APPARATUS

(71) Applicant: Murata Manufacturing Co., Ltd., Nagaokakyo (JP)

(72) Inventors: Nobuhisa Mizobe, Nagaokakyo-shi (JP); Makoto Oono, Nagaokakyo-shi (JP)

(21) Appl. No.: 17/165,285

(22) Filed: Feb. 2, 2021

Related U.S. Application Data
(63) Continuation of application No. PCT/JP2019/030858, filed on Aug. 6, 2019.

(30) Foreign Application Priority Data
Sep. 11, 2018 (JP) 2018-169765

(51) Int. Cl. A61B 5/11

(52) U.S. Cl. CPC A61B 5/1115; A61B 2562/0219; A61B 5/1116; A61B 5/1118

(57) ABSTRACT

A bed apparatus capable of detecting a state of a user. The bed apparatus includes a bedboard that supports a user, a bed frame that is attached to the bedboard, an acceleration sensor attached to the bedboard, and a controller that detects a state of the user based on a result of detection of the acceleration sensor. The acceleration sensor can detect accelerations in at least two axial directions and is attached to a surface of the bedboard such that two acceleration detection axes of the acceleration sensor are parallel to the surface of the bedboard.

### CROSS-REFERENCE TO RELATED APPLICATIONS

The present application is a continuation of International application No. PCT/JP2019/030858, filed Aug. 6, 2019, which claims priority to Japanese Application No. 2018-169765, filed on Sep. 11, 2018, the entire contents of each of which are incorporated herein by reference.

### TECHNICAL FIELD

The present disclosure relates to a sensor unit and a bed apparatus.

### BACKGROUND

In recent years, bed apparatuses used in medical facilities and other facilities have been required to detect the movement of a user on the bed apparatus, such as the sitting-up of the user. In one approach, a bed apparatus detects the load on a bed part and computes the position of the center of gravity of a user. In this approach, the bed apparatus may detect the sitting-up of a user on the basis of the movement of the computed position of the center of gravity of the user over time. The bed apparatus may include, at the four corners of a frame with legs supporting the bed part, four load sensors for detecting a load on the bed part and generating a load signal.

However, because a system such as the load sensors need to be embedded in the bed apparatus in advance (e.g., at the time of the manufacture of the bed apparatus), detection of the state of a user in an existing bed apparatus cannot be easily performed. Moreover, because in this approach the sensors are attached to the corners, there is no account for the change in center of gravity due to reclining.

### SUMMARY

To address the shortcomings of conventional bed apparatuses, the present disclosure describes a sensor unit and a bed apparatus capable of easily detecting the state of a user.

A bed apparatus according to an embodiment of the present disclosure which is capable of detecting a state of a user includes a bedboard that supports the user, a bed frame that is attached to the bedboard, an acceleration sensor attached to the bedboard, and a detection unit that detects a state of the user based on results acquired by the acceleration sensor. The acceleration sensor can detect accelerations in at least two axial directions and is attached to a surface of the bedboard such that two acceleration detection axes of the acceleration sensor are parallel to the surface of the bedboard.

A sensor unit according to an embodiment of the present disclosure which is attachable to a bed apparatus and detects a state of a user of the bed apparatus includes a sensor element that can detect accelerations in at least two axial directions and a fixing part with which the sensor unit is attached to a surface of a bedboard in the bed apparatus such that two acceleration detection axes of the sensor element are parallel to the surface of the bedboard.

According to the present disclosure, since two acceleration detection axes are parallel to the surface of a bedboard, the state of a user can be easily detected.

The above simplified summary of example aspects serves to provide a basic understanding of the present disclosure. This summary is not an extensive overview of all contemplated aspects, and is intended to neither identify key or critical elements of all aspects nor delineate the scope of any or all aspects of the present disclosure. Its sole purpose is to present one or more aspects in a simplified form as a prelude to the more detailed description of the disclosure that follows. To the accomplishment of the foregoing, the one or more aspects of the present disclosure include the features described and exemplarily pointed out in the claims.

### BRIEF DESCRIPTION OF THE DRAWINGS

The accompanying drawings, which are incorporated into and constitute a part of this specification, illustrate one or more example aspects of the present disclosure and, together with the detailed description, serve to explain their principles and implementations.

FIG. 1 is a schematic diagram of an example of a bed apparatus according to a first embodiment;

FIG. 2 is a block diagram of an example of a bed apparatus according to the first embodiment;

FIG. 3 is a schematic diagram of an example of a sensor unit according to the first embodiment;

FIG. 4(a) is a schematic diagram illustrating the state where a user is in a supine position on a bed apparatus according to the first embodiment;

FIG. 4(b) is a schematic diagram illustrating the state where a user is in a supine position on a bed apparatus from a side perspective according to the first embodiment;

FIG. 5(a) is a schematic diagram illustrating the state where a user is in a sitting-up state on a bed apparatus according to the first embodiment;

FIG. 5(b) is a schematic diagram illustrating the state where a user is in a sitting-up state on a bed apparatus from a side perspective according to the first embodiment;

FIG. 6(a) is a schematic diagram illustrating the state where a reclining function is used in a bed apparatus according to the first embodiment;

FIG. 6(b) is a schematic diagram illustrating the state where a reclining function is used to a different degree in a bed apparatus according to the first embodiment;

FIG. 7 is a flow chart illustrating an exemplary process of detecting the state of a user on a bed apparatus according to the first embodiment;

FIG. 8 is a schematic diagram of an example of a bed apparatus according to a second embodiment;

FIG. 9(a) is a schematic diagram illustrating the state where a user is in a sitting position with soles of feet on the floor on a bed apparatus according to the second embodiment;

FIG. 9(b) is a schematic diagram illustrating the state where a user is in a sitting position with soles of feet on the floor on a bed apparatus from a first side perspective according to the second embodiment;

FIG. 9(c) is a schematic diagram illustrating the state where a user is in a sitting position with soles of feet on the floor on a bed apparatus from a second side perspective according to the second embodiment.

### DETAILED DESCRIPTION

Embodiments of the present disclosure will be described in detail below with reference to the accompanying drawings. In the drawings, the same reference numeral is used to represent the same part or the corresponding part to avoid repeated explanation.

### First Embodiment

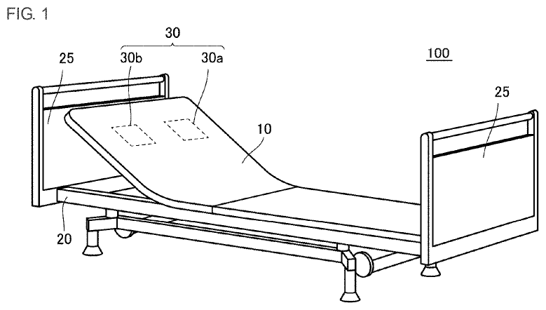

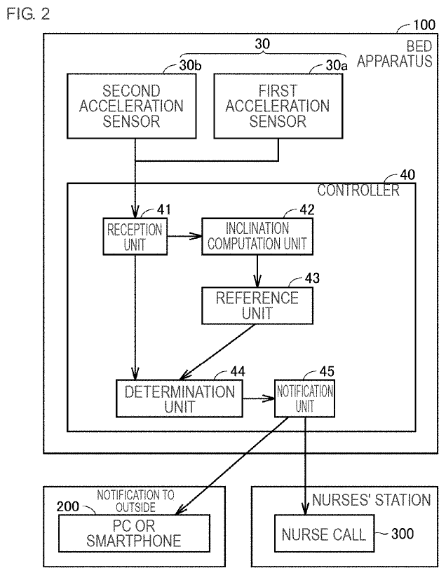

FIG. 1 is a schematic diagram of an example of a bed apparatus according to the first embodiment. FIG. 2 is a block diagram of an example of a bed apparatus according to the first embodiment. A bed apparatus 100 includes a bedboard 10 that supports a user and a bed frame 20 that supports the bedboard 10. Wall parts 25 are provided at the two short sides of the bed frame 20. On the bedboard 10 in the bed apparatus 100, a user can lie on the back with the head toward either of the wall parts 25. The state where a user lies on the back on the bed apparatus 100 is hereinafter also referred to as a supine position.

The bed apparatus 100 has a reclining function. The angle of a part of the bedboard 10 on the side of the head of a user with respect to the bed frame 20 can therefore be changed. The bed apparatus 100 may manually or electrically perform the reclining function. In the case where the reclining function is electrically performed, the bed apparatus 100 includes a motor (not illustrated), an actuator (not illustrated), and a linkage (not illustrated) in the bed frame 20.

The bed apparatus 100 further includes, on the bedboard 10, acceleration sensors 30 for detecting the state of a user. The acceleration sensors 30 are provided on the surface or the undersurface of the bedboard 10 opposite to the side of a user. In the example illustrated in FIG. 1, the two acceleration sensors 30 are provided on the bedboard 10: an acceleration sensor 30a (a first acceleration sensor) and an acceleration sensor 30b (a second acceleration sensor).

The acceleration sensor 30 may detect accelerations in at least two axial directions and may be of any type such as a capacitive type, a piezoresistive type, or a strain gauge type. The following description will be made on the assumption that the acceleration sensor 30 can detect accelerations in two axial directions.

The acceleration sensor 30 is configured as a sensor unit to be easily attached on the bedboard 10. FIG. 3 is a schematic diagram of an example of a sensor unit according to the first embodiment. The acceleration sensor 30 configured as a sensor unit includes a sensor element 31 for detecting an acceleration and a fixing part 32 that attaches the sensor element 31 to the surface of the bedboard 10. The fixing part 32 is plate-like in shape and is provided with the sensor element 31 on the surface thereof opposite to the surface thereof to be attached to the bedboard 10.

One of the two acceleration detection axes of the sensor element 31 is directed to the X direction illustrated in FIG. 3 and the other one of them is directed to the Y direction illustrated in FIG. 3. On the plane (X-Y plane) including the two acceleration detection axes, the sensor element 31 and the fixing part 32 are in contact with each other. The surface of the fixing part 32 to be attached to the bedboard 10 is therefore always parallel to the two acceleration detection axes of the sensor element 31. That is, only by attaching this surface of the fixing part 32 to the bedboard 10, the two acceleration detection axes are always parallel to the bedboard 10. The two acceleration detection axes and the surface of the bedboard 10 do not necessarily have to be perfectly parallel to each other, for example, within the allowable range of detection of the acceleration sensor 30.

As illustrated in FIG. 2, a detection signal generated by the acceleration sensor 30 is transmitted to a controller 40 in a wired or wireless manner. The controller 40 includes a reception unit 41, an inclination computation unit 42, a reference unit 43, a determination unit 44, and a notification unit 45.

The controller 40 is provided with a CPU (central processing unit), a ROM (read-only memory) storing programs and control data that the CPU uses to operate, a RAM (random access memory) functioning as a work area for the CPU, an input/output interface for maintaining signal consistency with a peripheral, and other components. The controller 40 executes, for example, programs and control data, thereby functioning as the reception unit 41, the inclination computation unit 42, the reference unit 43, the determination unit 44, and the notification unit 45.

A detection signal transmitted from the acceleration sensor 30 is received by the reception unit 41. The detection signal received by the reception unit 41 is transmitted to the inclination computation unit 42 and the reference unit 43. The inclination computation unit 42 computes the angle of inclination of the bedboard 10 on the basis of the detection signal generated by the acceleration sensor 30. For example, the inclination computation unit 42 computes the angle of inclination of the bedboard 10 on the basis of an acceleration in a direction vertical to the surface of the bedboard 10 when the bedboard 10 is reclined.

The determination unit 44 detects the state of a user on the basis of the detection signal (detection result) generated by the acceleration sensor 30. That is, the controller 40 including the determination unit 44 functions as a detection unit for detecting the state of a user on the basis of the detection result of the acceleration sensor 30. For example, the determination unit 44 detects the fact that a user in the supine position on the bed apparatus 100 has lifted the user's head from the bedboard 10 and gone into the sitting-up state.

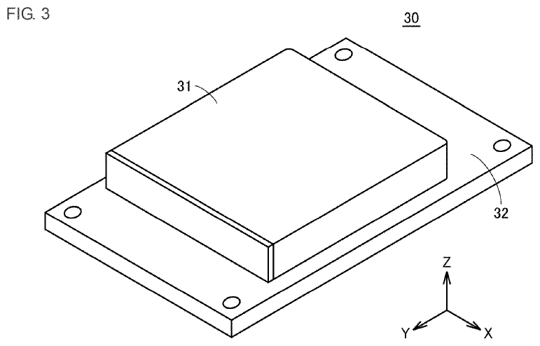

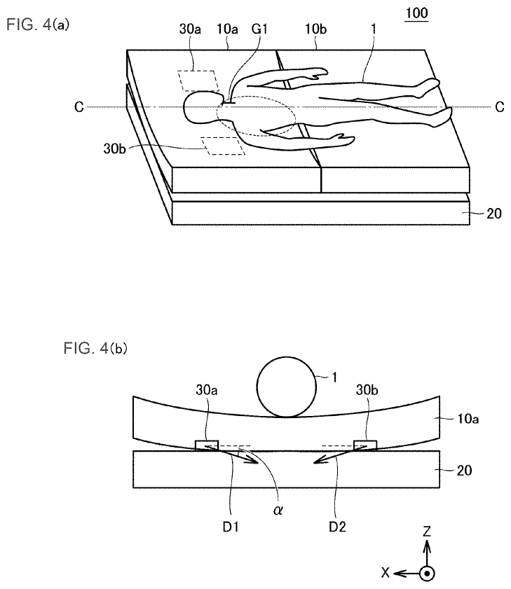

The principle that the determination unit 44 detects the state of a user will be described with reference to the accompanying drawings. FIG. 4 comprises two schematic diagrams illustrating the state where a user 1 is in a supine position on a bed apparatus according to the first embodiment. FIG. 4(a) is a perspective view of the bed apparatus 100 on which the user 1 is in the supine position. FIG. 4(b) is a side view of the bed apparatus 100 on which the user 1 is in the supine position when the bed apparatus 100 is viewed from the side of the head of the user 1.

As illustrated in FIG. 4(a), the bedboard 10 is divided into a board part 10a on the side of the head of the user 1 and a board part 10b on the side of the feet of the user 1. As shown in FIG. 4(a), part 10a may be half of bedboard 10 and part 10b may be half of bedboard 10. In the board part 10a, the acceleration sensors 30a and 30b are provided. When the user 1 is in the supine position, the load of the user 1 is mainly applied to the board part 10a. Accordingly, by providing the acceleration sensors 30a and 30b in the board part 10a, the acceleration sensors 30a and 30b can detect the strain component of the bedboard 10.

Referring to FIG. 4(b), the acceleration sensors 30a and 30b provided on the undersurface of the board part 10a detect strain components D1 and D2 of the bedboard 10 strained by the load of the user 1. Specifically, the acceleration sensors 30a and 30b detect the strain components D1 and D2 of the bedboard 10 generated as a result of the supine position of the user 1 as acceleration components inclined at an angle α from the horizontal direction in the drawing. The strain components of the bedboard 10 can be obtained by performing a vector operation upon DC components detected on the two respective acceleration detection axes of the acceleration sensors 30a and 30b which are parallel to the plane of the bedboard 10. Although the acceleration sensors 30a and 30b are embedded in the undersurface of the board part 10a in FIG. 4(b), the acceleration sensors 30a and 30b may be attached to the undersurface of the board part 10a.

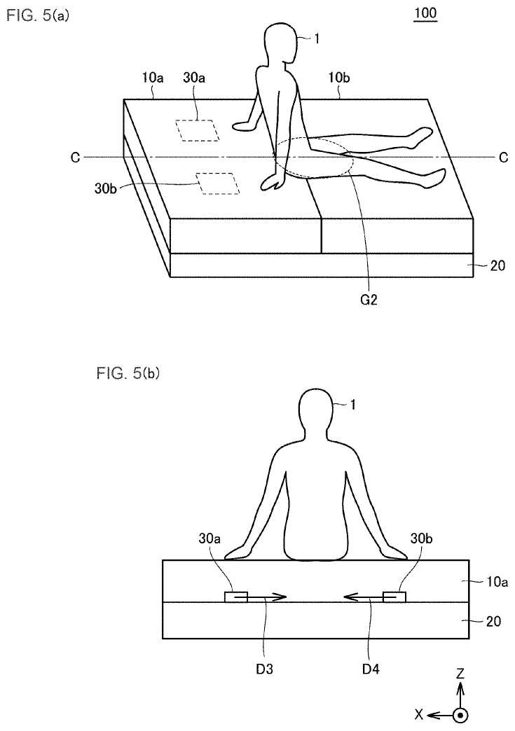

FIG. 5 comprises two schematic diagrams illustrating the state where the user 1 is in the sitting-up state on the bed apparatus 100 according to the first embodiment. FIG. 5(a) is the perspective view of the bed apparatus 100 on which the user 1 is in the sitting-up state. FIG. 5(b) is the side view of the bed apparatus 100 on which the user 1 is in the sitting-up state when the bed apparatus 100 is viewed from the side of the head of the user 1.

As illustrated in FIG. 5(a), the load of the user 1 is not applied to the board part 10a when the user 1 is in the sitting-up state. Accordingly, the amount of the strain component of the bedboard 10 detected by the acceleration sensors 30a and 30b becomes small. Specifically, when the user 1 goes into the sitting-up state, the strain of the bedboard 10 is eliminated and the acceleration sensors 30a and 30b detect strain components D3 and D4 of the bedboard 10 as acceleration components (DC components) in the horizontal direction as illustrated in FIG. 5(b).

The determination unit 44 uses the information of the reference unit 43 illustrated in FIG. 2 to determine whether the user 1 is in the supine position or the sitting-up state on the basis of the strain component of the bedboard 10 detected by the acceleration sensors 30a and 30b. For example, the reference unit 43 stores a threshold value used for the determination of whether the user 1 is in the supine position or the sitting-up state. When the strain component of the bedboard 10 detected by the acceleration sensors 30a and 30b is greater than the threshold value obtained from the reference unit 43, the determination unit 44 determines that the user 1 is in the supine position.

For example, the reference unit 43 may store an algorithm used for the determination of whether the user 1 is in the supine position or the sitting-up state. The determination unit 44 determines whether the user 1 is in the supine position or the sitting-up state using the algorithm obtained from the reference unit 43. Specifically, as the algorithm used for the determination of the state of the user 1, a function is considered with which a determination value is calculated as a result of the input of the strain component of the bedboard 10 detected by the acceleration sensors 30a and 30b. In this function, the change from a position of the center of gravity G1 of the user 1 (FIG. 4(a)) to a position of the center of gravity G2 of the user 1 (FIG. 5(a)), which occurs when the posture of the user 1 changes from the supine position to the sitting-up state, is provided in advance in the form of a function as the change in the strain component of the bedboard 10.

The case will be described where the angle of inclination of the board part 10a is changed by the reclining function. When the angle of inclination of the board part 10a changes, the load of the user 1 applied to the board part 10a changes. Accordingly, a threshold value or an algorithm that the determination unit 44 uses to determine whether user 1 is in the supine position or the sitting-up state also changes. The reference unit 43 therefore stores threshold values or algorithms corresponding to the angles of inclination of the board part 10a.

In the bed apparatus 100 according to the first embodiment, the state of the user 1 can be detected on the basis of the strain component of the board part 10a detected by the acceleration sensors 30a and 30b while the angle of inclination of the board part 10a can be obtained on the basis of the detection signal of the acceleration sensor 30. That is, in the bed apparatus 100, a plurality of matters, the angle of inclination of the board part 10a and the state of the user 1 (e.g., the sitting-up state), can be detected only by the acceleration sensor 30.

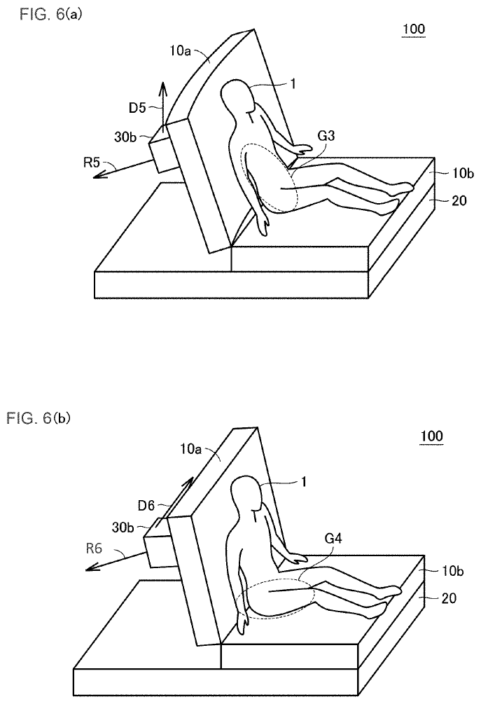

The principle that the determination unit 44 detects the state of a user when the reclining function is used in the bed apparatus 100 will be described with reference to the accompanying drawings. FIG. 6 comprises two schematic diagrams illustrating the state where the reclining function is used in the bed apparatus 100 according to the first embodiment. FIG. 6(a) illustrates the state where the reclining function is used and the user 1 leans against the board part 10a on the bed apparatus 100. FIG. 6(b) illustrates the state where the reclining function is used and the user 1 sits up on the bed apparatus 100 (e.g., reclines to a different degree than shown in FIG. 6(a).

Since the user 1 leans against the board part 10a in FIG. 6(a), the acceleration sensors 30a and 30b can detect a strain component D5 of the board part 10a. Since the load of the user 1 applied to the board part 10a when the reclining function is used is smaller than that when the user 1 is in the supine position, the amount of the strain component D5 of the board part 10a is smaller than the amounts of the strain components D1 and D2 illustrated in FIG. 4(b). Although only the acceleration sensor 30b is illustrated in FIG. 6(a), the acceleration sensor 30a is provided on the rear side of the board part 10a relative to the plane of the drawing.

Since the user 1 sits up and does not lean against the board part 10a in FIG. 6(b), the acceleration sensors 30a and 30b detect a strain component D6 of the board part 10a. The amount of the strain component D6 of the bedboard 10 in which the load of the user 1 is not applied to the board part 10a is smaller than the amount of the strain component D5. Although only the acceleration sensor 30b is illustrated in FIG. 6(b), the acceleration sensor 30a is provided on the rear side of the board part 10a relative to the plane of the drawing.

When the reclining function is used, the inclination computation unit 42 computes the angle of inclination of the bedboard 10 using inclination components R5 and R6 of the board part 10a obtained as the detection signals of the acceleration sensors 30a and 30b. The inclination computation unit 42 inputs the computed angle of inclination of the bedboard 10 into the reference unit 43 (see FIG. 2). The reference unit 43 provides information used for the determination of whether a user is in the sitting-up state for the determination unit 44 in accordance with the angle of inclination of the bedboard 10.

For example, the reference unit 43 stores threshold values corresponding to the angles of inclination of the bedboard 10 which are used for the determination of whether the user 1 is in the sitting-up state. Accordingly, the reference unit 43 provides a threshold value for the determination unit 44 in accordance with the angle of inclination of the bedboard 10 obtained from the inclination computation unit 42. The determination unit 44 determines that the user 1 leans against the board part 10a when the strain component of the bedboard 10 detected by the acceleration sensors 30a and 30b is greater than the threshold value corresponding to the angle of inclination of the bedboard 10.

For example, the reference unit 43 may store an algorithm taking the angle of inclination of the bedboard 10 into consideration which is used for the determination of whether the user 1 is in the sitting-up state. The determination unit 44 determines whether the user 1 is in the sitting-up state on the basis of the algorithm taking the angle of inclination of the bedboard 10 into consideration. Specifically, as the algorithm used for the determination of the state of the user 1, a function is considered with which a determination value is calculated as a result of the input of the strain component of the bedboard 10 detected by the acceleration sensors 30a and 30b. In this function, for each angle of inclination of the bedboard 10, the change from a center of gravity G3 of the user 1 (FIG. 6(a)) to a center of gravity G4 of the user 1 (FIG. 6(b)), which occurs when the posture of the user 1 changes from the state where the user 1 leans against the board part 10a to the sitting-up state, is provided in advance in the form of a function as the change in the strain component of the bedboard 10.

The number of the acceleration sensors 30 provided at the bedboard 10 and the position of the acceleration sensor 30 will be described. In the case where the bed apparatus 100 has the reclining function, the bedboard 10 is divided into the board part 10a supporting the upper half of the body of the user 1 and the board part 10b supporting the lower half of the body of the user 1. Since the amount of upper-body load of the user 1 is large when the user 1 is in the supine position, it is desired that the acceleration sensor 30 be provided at the board part 10a to allow the acceleration sensor 30 to obtain the strain component of the bedboard 10 on the upper-body side.

It is desired that the acceleration sensor 30 be provided at a position where the acceleration sensor 30 can detect the state of the user 1 best. The position where the state of the user 1 can be detected best is a position where the bedboard 10 strains most when the user 1 is in the supine position. That is, as described above, the highest load is applied to a position that is in contact with the upper half of the body of the user 1 when the user 1 is in the supine position and the board part 10a strains from the position in an arc. Accordingly, by providing the single acceleration sensor 30 at the position, the state of the user 1 can be detected.

However, the position to which the highest load of the user 1 is applied changes when the user 1 turns over or moves on the bedboard 10. It is therefore desired that the multiple acceleration sensors 30 be provided on the surface of the bedboard 10. As illustrated in FIG. 4(b), by providing the two acceleration sensors 30a and 30b on the undersurface of the bedboard 10 (in particular, the board part 10a), the bed apparatus 100 is allowed to accurately detect the sitting-up state of the user 1.

In the case where the acceleration sensor 30 is provided along the center line (e.g., line C-C in FIG. 4(a)) of the short side of the bedboard 10, the bed apparatus 100 may not be able to accurately detect the sitting-up state of the user 1 when the user 1 is not at the center of the bedboard 10. It is therefore desired that the acceleration sensor 30 be provided at a position outside the center line of the short side of the bedboard 10. By providing the two acceleration sensors 30a and 30b on both sides of the center line of the short side of the bedboard 10, the occurrence of the case can be prevented where the bed apparatus 100 cannot detect the state of the user 1 when the user 1 is not at the center of the bedboard 10.

It is further desired that the multiple acceleration sensors 30a and 30b be provided at positions that are symmetric with respect to the center line of the short side of the bedboard 10. Using acceleration components obtained by the acceleration sensors 30a and 30b provided at positions that are symmetric with respect to the center line of the short side of the bedboard 10, the bed apparatus 100 can easily perform computation for the determination of the state of a user.

Subsequently, the notification unit 45 illustrated in FIG. 2 externally outputs the state of the user 1 that is the determination result of the determination unit 44. Specifically, the notification unit 45 wirelessly transmits the determination result of the determination unit 44 to a personal computer (PC) or a smartphone 200 to provide the notification of the state of the user 1 or connects to a nurse call 300 to notify a nurses' station that the user 1 is in the sitting-up state. Alternatively, the notification unit 45 may provide the notification that the user 1 is in the sitting-up state using an LED buzzer (not illustrated) provided at the controller 40.

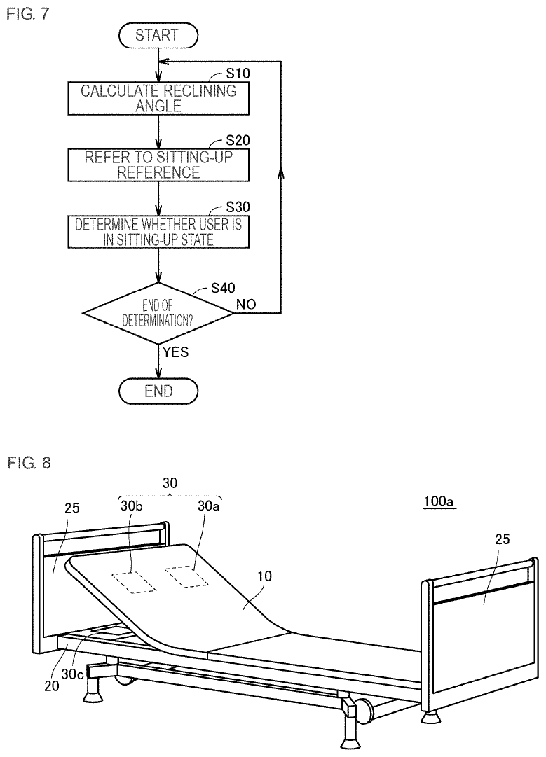

Next, the process of detecting the state of the user 1 in the bed apparatus 100 will be described with reference to a flow chart. FIG. 7 is a flow chart illustrating an exemplary process of detecting the state of a user in the bed apparatus 100 according to the first embodiment. At step S10, the determination unit 44 calculates a reclining angle (the angle of inclination of the board part 10a) on the basis of the detection results of the acceleration sensors 30a and 30b.

At step S20, the determination unit 44 reads a sitting-up reference (e.g., a threshold value or an algorithm) corresponding to the reclining angle (the angle of inclination of the board part 10a) calculated in step S10 from the reference unit 43 and refers to the sitting-up reference.

At step S30, the determination unit 44 determines whether the user 1 is in the sitting-up state on the basis of the sitting-up reference that has been referred to in step S20. Subsequently, the determination unit 44 receives a signal indicating whether determination is to end (step S40). When receiving a signal indicating that determination is to end (YES in step S40), the determination unit 44 ends the determination of whether the user 1 is in the sitting-up state. On the other hand, when not receiving the signal indicating that determination is to end (NO in step S40), the determination unit 44 continues the determination of whether the user 1 is in the sitting-up state and the process returns to step S10.

As described above, the bed apparatus 100 according to the first embodiment can detect the state of the user 1. The bedboard 10 includes the bedboard 10 that supports the user 1, the bed frame 20 that supports the bedboard 10, the acceleration sensor 30 attached to the bedboard 10, and the controller 40 (a detection unit) that detects a state of the user based on a result of detection of the acceleration sensor 30. The acceleration sensor 30 can detect accelerations in at least two axial directions and is attached to a surface of the bedboard 10 such that two acceleration detection axes of the acceleration sensor 30 are parallel to the surface of the bedboard. The bed apparatus 100 having this configuration can easily detect the state of a user, even if an existing bed apparatus is used.

The multiple acceleration sensors 30a and 30b may be attached to the surface of the bedboard 10. With this configuration, the bed apparatus 100 can accurately detect the sitting-up state of the user 1.

The multiple acceleration sensors 30a and 30b may be provided at a position outside a center line of a short side of the bedboard 10. With this configuration, even if the user 1 is not located at the center of the bedboard 10, the bed apparatus 100 can easily detect the state of the user 1.

The multiple acceleration sensors 30a and 30b may be provided at positions that are symmetric with respect to the center line of the short side of the bedboard 10. With this configuration, the bed apparatus 100 can easily perform computation for the determination of the state of a user on the basis of acceleration components obtained from the acceleration sensors 30a and 30b.

The acceleration sensors 30a and 30b are attached to a surface (undersurface) of the bedboard 10 opposite to another surface of the bedboard 10 supporting the user 1. With this configuration, the acceleration sensors 30a and 30b can be attached to an existing bed apparatus and detect the state of the user 1. The acceleration sensors 30a and 30b may be embedded in the bedboard 10 or may be attached to the surface of the bedboard 10 supporting the user 1. Alternatively, the acceleration sensors 30a and 30b may be attached to a mattress mounted on the bedboard 10.

In the bed apparatus 100, the bedboard 10 may have the board part 10a (a movable part) that can be partly raised. That is, the bed apparatus 100 may have the reclining function. In the case where the bed apparatus 100 has the reclining function, the acceleration sensors 30a and 30b are attached to the board part 10a (the movable part). With this configuration, the bed apparatus 100 having the reclining function can also detect the state of the user 1.

The controller 40 (the detection unit) may detect the state of the user 1 and the angle of inclination (a position) of the board part 10a (the movable part) based on results of detection of the acceleration sensors 30a and 30b. With this configuration, the bed apparatus 100 can detect a plurality of matters, the angle of inclination of the board part 10a and the state of the user 1 (e.g., the sitting-up state) using only the acceleration sensor 30. In addition, the bed apparatus 100 can detect the state of the user 1 (e.g., the sitting-up state) in consideration of the angle of inclination of the board part 10a.

The acceleration sensor 30 attached to the bedboard 10 may be configured as a sensor unit. The sensor unit is the acceleration sensor 30 that is attachable to the bed apparatus 100 and detects the state of the user 1 of the bed apparatus 100. The sensor unit includes the sensor element 31 that can detect accelerations in at least two axial directions and the fixing part 32 with which the sensor unit is attached to a surface of the bedboard 10 in the bed apparatus 100 such that two acceleration detection axes of the sensor element 31 are parallel to the surface of the bedboard. With this configuration, the acceleration sensor 30 can be easily attached to the bedboard 10 in the existing bed apparatus 100.

### Second Embodiment

In the first embodiment, the configuration has been described in which the acceleration sensors 30 are attached to the bedboard 10. In the second embodiment, the configuration will be described in which acceleration sensors are attached not only to a bedboard but also to a bed frame. FIG. 8 is a schematic diagram of an example of a bed apparatus 100a according to the second embodiment. In the bed apparatus 100a illustrated in FIG. 8, the same configurations as those of the bed apparatus 100 illustrated in FIGS. 1 and 2 will be denoted by the same reference numerals, and a detailed description thereof will not be repeated.

The bed apparatus 100a includes the bedboard 10 that supports a user and the bed frame 20 that supports the bedboard 10. The wall parts 25 are provided at the two short sides of the bed frame 20. The bed apparatus 100a has a reclining function. The angle of a part of the bedboard 10 on the side of the head of a user with respect to the bed frame 20 can therefore be changed.

The bed apparatus 100a further includes, at the bedboard 10 and the bed frame 20, the acceleration sensors 30 for detecting the state of a user. Among the acceleration sensors 30, the acceleration sensors 30a and 30b are provided on the surface (undersurface) of the bedboard 10 opposite to the side of a user. Among the acceleration sensors 30, an acceleration sensor 30c is attached to the bed frame 20.

The acceleration sensors 30a, 30b, and 30c may detect accelerations in at least two axial directions and may be of any type such as a capacitive type, a piezoresistive type, or a strain gauge type. The following description will be made on the assumption that the acceleration sensors 30a, 30b, and 30c can detect accelerations in two axial directions.

As described in the first embodiment, the acceleration sensors 30a and 30b detect the state of the user 1, especially, the strain component of the bedboard 10 used for the determination of whether the user 1 is in the sitting-up state. However, in the case where the user 1 rises and sits on the edge of the bed apparatus 100a, that is, is in the sitting position with soles of feet on the floor, the acceleration sensors 30a and 30b on the side of the upper half of the body of the user 1 cannot detect the strain component of the bedboard 10. In order to enable the determination of whether the user 1 is in the sitting position with soles of feet on the floor, the acceleration sensor 30c is attached to the bed frame 20.

The acceleration sensor 30c detects the inclination of the bed frame 20 which changes with the position of the user 1 on the bed apparatus 100a. Like detection signals obtained by the acceleration sensors 30a and 30b, a detection signal obtained by the acceleration sensor 30c is received by the reception unit 41 in the controller 40 and is used for the determination of whether the user 1 is in the sitting position with soles of feet on the floor in the determination unit 44.

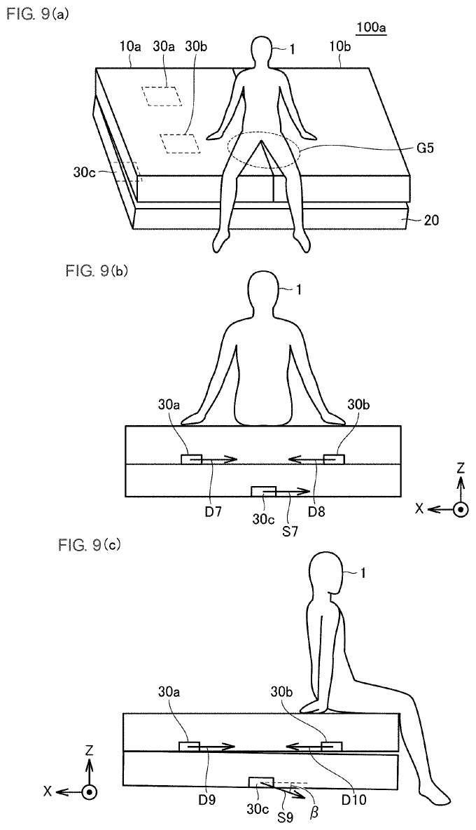

The configuration will be described in detail with reference to the drawings in which the determination unit 44 determines whether the user 1 is in the sitting position with soles of feet on the floor. FIG. 9 comprises three schematic diagrams illustrating the state where the user 1 is in the sitting position with soles of feet on the floor on the bed apparatus 100a according to the second embodiment. FIG. 9(a) is a perspective view of the bed apparatus 100a on which the user 1 is in the sitting position with soles of feet on the floor. FIG. 9(b) is a side view of the bed apparatus 100a on which the user 1 in the sitting-up state when the bed apparatus 100a is viewed from the side of the head of the user 1. FIG. 9(c) is a side view of the bed apparatus 100a on which the user 1 is in the sitting position with soles of feet on the floor when the bed apparatus 100a is viewed from the side of the head of the user 1.

As illustrated in FIG. 9(a), the acceleration sensors 30a and 30b are attached to the board part 10a and the acceleration sensor 30c is attached to the bed frame 20. When the user 1 is in the sitting position with soles of feet on the floor, the load of the user 1 is mainly applied to one end of the short side of the bedboard 10 around the center part of the bedboard 10 (the boundary between the board parts 10a and 10b). That is, the position of the center of gravity of the user 1 is a position of the center of gravity G5 illustrated in FIG. 9(a).

Accordingly, when the user 1 is in the sitting position with soles of feet on the floor, the board part 10a does not strain because of the load of the user 1 and the acceleration sensors 30a and 30b cannot detect the strain component of the bedboard 10. On the other hand, since the bed frame 20 inclines because of the load of the user 1, the inclination component of the bed frame 20 can be detected by the acceleration sensor 30c provided at the bed frame 20. The acceleration sensor 30c can detect the inclination component of the bed frame 20 irrespective of the installation position thereof because the bed frame 20 is integrally formed.

For comparison, FIG. 9(b) illustrates strain components D7 and D8 of the bedboard 10 detected by the acceleration sensors 30a and 30b and an inclination component S7 of the bed frame 20 detected by the acceleration sensor 30c when the user 1 is in the sitting-up state. When the user 1 goes into the sitting-up state, the strain of the bedboard 10 is eliminated and the strain components D7 and D8 of the bedboard 10 are detected as acceleration components (DC components) in the horizontal direction in the drawing. Since the user 1 is located at the substantially center of the short side of the bed frame 20 when the user 1 is in the sitting-up state, the bed frame 20 is nearly horizontal. Accordingly, the inclination component S7 of the bed frame 20 is detected as an acceleration component (DC component) in the horizontal direction in the drawing.

FIG. 9(c) illustrates strain components D9 and D10 of the bedboard 10 detected by the acceleration sensors 30a and 30b and an inclination component S9 of the bed frame 20 detected by the acceleration sensor 30c when the user 1 is in the sitting position with soles of feet on the floor. When the user 1 goes into the sitting position with soles of feet on the floor, the bedboard 10 does not strain and the strain components D9 and D10 of the bedboard 10 are detected as acceleration components (DC components) in the horizontal direction in the drawing. Since the user 1 is located at one end of the short side of the bed frame 20 when the user 1 is in the sitting position with soles of feet on the floor, the bed frame 20 is inclined toward the user. Accordingly, the inclination component S9 of the bed frame 20 is detected as an acceleration component inclined at an angle β from the horizontal direction in the drawing.

The determination unit 44 uses the information of the reference unit 43 illustrated in FIG. 2 to determine whether the user 1 is in the sitting position with soles of feet on the floor or the sitting-up state on the basis of the inclination component S9 of the bed frame 20 detected by the acceleration sensor 30c. For example, the reference unit 43 stores a threshold value used for the determination of whether the user 1 is in the sitting position with soles of feet on the floor or the sitting-up state. When the inclination component S9 of the bed frame 20 detected by the acceleration sensor 30c is greater than the threshold value obtained from the reference unit 43, the determination unit 44 determines that the user 1 is in the sitting position with soles of feet on the floor. The reference unit 43 may store an algorithm used for the determination of whether the user 1 is in the sitting position with soles of feet on the floor or the sitting-up state. The determination unit 44 may determine whether the user 1 is in the sitting position with soles of feet on the floor on the basis of the algorithm.

As described above, one of the multiple acceleration sensors 30 (e.g., the acceleration sensor 30c) is attached to the bed frame 20 in the bed apparatus 100a according to the second embodiment. As a result, the bed apparatus 100a can accurately detect that the user 1 is in the sitting position with soles of feet on the floor.

In the above embodiments, the bed apparatuses in which the bedboard 10 and the bed frame 20 are separately provided have been described. However, the configurations described in the above embodiments can be applied to a bed apparatus in which a bedboard and a bed frame are integrally formed.

The embodiments disclosed herein are illustrative only and are not intended to be limiting in any way. The scope of the present disclosure is defined by the appended claims rather than the foregoing description, and it should be understood that all the changes conceived from the meaning and scope of the claims and their equivalents are included in the scope of the present disclosure.

1. A bed apparatus capable of detecting a state of a user, the bed apparatus comprising:
a bedboard that supports the user;
a bed frame attached to the bedboard;
an acceleration sensor configured to detect accelerations in at least two axial directions, and which is attached to a surface of the bedboard such that two acceleration detection axes of the acceleration sensor are parallel to the surface of the bedboard; and
a detection unit that detects the state of the user based on results acquired by the acceleration sensor.

2. The bed apparatus according to claim 1, wherein the acceleration sensor is one of a plurality of acceleration sensors attached to the surface of the bedboard.

3. The bed apparatus according to claim 2, wherein the plurality of acceleration sensors are provided at positions that are symmetric with respect to a center line of a short side of the bedboard.

4. The bed apparatus according to claim 1, wherein the acceleration sensor is provided at a position outside a center line of a short side of the bedboard.

5. The bed apparatus according to claim 1, wherein the acceleration sensor is attached to a surface of the bedboard opposite to another surface of the bedboard for supporting the user.

6. The bed apparatus according to claim 1, wherein the bedboard has a movable part that can be partly raised, and
wherein the acceleration sensor is attached to the movable part.

7. The bed apparatus according to claim 6, wherein the detection unit detects the state of the user and a position of the movable part based on the results acquired by the acceleration sensor.

8. The bed apparatus according to claim 7, wherein the detection unit detects the state of the user and a position of the movable part based on only the results acquired by the acceleration sensor.

9. The bed apparatus according to claim 6, wherein the detection unit determines an angle of inclination of the movable part, and wherein the detection unit detects the state of the user based on both the results acquired by the acceleration sensor and the angle of inclination.

10. The bed apparatus according to claim 9, wherein the detection unit detects the state of the user by comparing the results acquired by the acceleration sensor against a threshold value corresponding to the angle of inclination, wherein a plurality of threshold values each corresponding to a respective angle of inclination is stored by the detection unit.

11. The bed apparatus according to claim 1, wherein the detection unit detects the state of the user based on a change in a center of gravity of the user from a first position on the bed apparatus to a second position on the bed apparatus.

12. The bed apparatus according to claim 1, wherein the acceleration sensor is embedded in the bedboard.

13. The bed apparatus according to claim 2, wherein at least one of the plurality of acceleration sensors is attached to the bed frame.

14. A sensor unit that is attachable to a bed apparatus and detects a state of a user of the bed apparatus, the sensor unit comprising:
a sensor element that can detect accelerations in at least two axial directions; and
a fixing part with which the sensor unit is attached to a moveable surface of the bed apparatus such that two acceleration detection axes of the sensor element are parallel to the moveable surface of the bed apparatus.

15. The sensor unit according to claim 14, wherein the sensor unit is further integrated with a plurality of sensor units attached to the moveable surface of the bed apparatus.
